(19) United States
(12) Patent Application Publication
(10) Pub. No.: US 2009/0017146 A1
(43) Pub. Date: Jan. 15, 2009

(54) Beta Vulgaris-Based Products

(76) Inventor: Yossi Shevach, Tel-Aviv (IL)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 10/596,959

(22) PCT Filed: Dec. 29, 2004
(86) PCT No.: PCT/IL04/01179

§ 371 Date: Jun. 30, 2006

Related U.S. Application Data
(60) Provisional application No. 60/533,709, filed on Dec. 31, 2003.

(51) Int. Cl. A61K 36/36; A61P 15/00; A61P 17/00; A61P 7/00

(52) U.S. Cl. 424/773

(57) ABSTRACT

Products based on beta vulgaris and especially red beet and methods for producing the same are provided. The orally administered beta vulgaris based products are adapted to be used for reducing homocysteine levels as well as reducing the cholesterol levels in the blood among other therapeutic effects.

### FIELD OF THE INVENTION

The present invention relates to products based on beta vulgaris. More particularly, the present invention relates to products such as beverages, juices, seasonings, vinegar, or delicacies based especially on red beet, as well as products for external use.

### BACKGROUND OF THE INVENTION

Beta vulgaris and especially red beet had been shown to contain substances such as folic acid, betain, and vitamin B6 that facilitate in balancing the levels of homocysteine in the blood. Reducing and normalization of the homocysteine levels by those three substances was shown by Lalouschek et al. in 1996 and Sella & Dolman in 1999. Red beet had been also used as treatment for high blood pressure (Krispil 2000) and had been shown to reduce the blood pressure and the LDL cholesterol levels in the blood of patients treated with a sugar beet fiber reach diet (Hagander et al., 1989). Red beet had been shown also to act as an antioxidant (Kahkonen et al. 1999; Kujala et al. 2000) and had been ranked as one of the 10 leading vegetables having antioxidation effect (Kujala et al. 2000).

Another component that has been shown to be in substantial amounts relative to other vegetables are phytostrols. Phytostrols from a vegetable source had been shown by Jones et al. to reduce the cholesterol level in the blood.

Red beet contains also fibers that had been shown to increase the activity of Glutathion peroxidase in the liver of rats. In rats fed with a diet containing 15% isolated fibers of red beet, the activity of the enzyme was increased (Bobek et al. 2000) wherein the enzyme is known for its antioxidant activity in cells and membranes (Shils et al.).

Beta vulgaris and especially red beet was provided also as supportive treatment in liver diseases and in fatty liver by PDR in 1999 and Weiss in 1988.

It should be emphasized that other components having therapeutic effect were found to be present in red beet.

The effects shown herein are attributed to the natural compositions of the substances in the red beet root or leaves and red beet has to be consumes as natural as possible in order to utilize its components in order to reduce the cholesterol and the homocysteine levels in the blood as well as to improve liver functionality. However, red beet is a vegetable that is hard to process to an edible form without fully or partially eliminating the active components. Moreover, red beet has a taste that is not favorable by many populations. Therefore, has to be processed in an alternative manner so as to become favorable to populations that are not familiar with the taste of red beet or are not used to consume red beet.

### SUMMARY OF THE INVENTION

It is an object of the present invention to provide edible and non edible products that are based on beta vulgaris containing components having antioxidant effect among other therapeutic effects, and methods to produce the same.

It is another object of the present invention to provide alcoholic and non-alcoholic beverages that are based on beta vulgaris, and methods to produce the same.

It is yet another object of the present invention to provide products based on red beet that are provided with other additives based on other herbs as well as sweeteners, food supplements etc. that can be used as well, so as to improve the effect of the products in the body.

In addition, it is an object of the present invention to provide beta voulgaris and especially red beet-based products in the form of beverages or paste that can be provided as food supplements.

It is therefore provided in accordance with a preferred embodiment of the present invention a method of reducing homocysteine levels as well as reducing the cholesterol levels in the blood among other therapeutic effects by orally administering a subject with beta vulgaris-based composition prepared by a process comprising:
preparing juice from beta vulgaris root;
adding sweetening agent;
filtering the resulting juice,

Furthermore, in accordance with another preferred embodiment of the present invention, the process further comprising fermenting the mixture.

Furthermore, in accordance with another preferred embodiment of the present invention, the process further comprising adding carbon dioxide.

Furthermore, in accordance with another preferred embodiment of the present invention, the process further comprising adding yeasts and fermenting the resulting juice.

Furthermore, in accordance with another preferred embodiment of the present invention, said beta vulgaris root is pilled.

Furthermore, in accordance with another preferred embodiment of the present invention, said beta vulgaris is red beet.

Furthermore, in accordance with another preferred embodiment of the present invention, said sweetening agent is selected from a group comprising fruit extract (preferably apple extract), beet molasses, sugar cane molasses, honey, maltose, brown or white sugar, sweet pomegranate extract, oligofructose, oligosaccharides.

Furthermore, in accordance with another preferred embodiment of the present invention, the process further comprising fermenting the resulting juice; allowing natural acidation; filtering the juice; and adding bacteria so as to produce vinegar or seasoning.

Furthermore, in accordance with another preferred embodiment of the present invention, the process further comprising heating the juice; adding gelling additive; steering the mixture until the mixture is fully homogenous; and cooling the mixture so as to produce jelly.

Furthermore, in accordance with another preferred embodiment of the present invention, said gelling additive is selected of a group comprising agar, tragacanth gum, gelatine, or pectin.

Furthermore, in accordance with another preferred embodiment of the present invention, the process further comprising adding lemon juice.

Furthermore, in accordance with another preferred embodiment of the present invention, the process further comprising adding oil and adding lechitin so as to produce paste adapted for spreading.

Furthermore, in accordance with another preferred embodiment of the present invention, said oil is olive oil.

Furthermore, in accordance with another preferred embodiment of the present invention, the process further comprising adding medical herbs.

It is further provided in accordance with another preferred embodiment of the present invention, a method of reducing homocysteine levels as well as reducing the cholesterol levels in the blood among other therapeutic effects by orally administering a subject with beta vulgaris-based composition prepared by a process comprising:
preparing juice from beta vulgaris root;
drying said juice into powder;

Furthermore, in accordance with another preferred embodiment of the present invention, the process further comprising encapsulating said powder.

Furthermore, in accordance with another preferred embodiment of the present invention, said beta vulgaris is a red beet.

In addition, it is provided in accordance with yet another preferred embodiment of the present invention, a method of reducing effects of gynecologic problems such as menopause, PMS, and fertility problems by orally administering a female with red beet-based composition comprising:
dry red beet extract;
dry pomegranate extract;
additives selected of a group comprising Cimicifuga racemosa, fatty acid—gla, ferulic acid, chamaelirium luteum, and Vitex agnus castus.

Further, in accordance with an additional embodiment of the present invention, a method of increasing the blood count of a subject, especially a subject going through a chemotherapy treatment, by orally administering the subject with red beet-based composition comprising:
dry red beet extract;
dry Astragalus membranaceus extract;

Yet, in accordance with another preferred embodiment of the present invention, a method of increasing the blood count of a subject, especially a subject going through a chemotherapy treatment, by orally administering the subject with red beet-based composition comprising:
dry red beet extract;
dry Withania somniferum extract;

It is also provided in accordance with another preferred embodiment of the present invention, a method of treating skin diseases such as seborrhea and seborrheic dermatitis by externally spreading the subject with products adapted for external use comprising juice extracted from beta vulgaris root.

Furthermore, in accordance with another preferred embodiment of the present invention, said beta vulgaris is a red beet.

additionally, in accordance with another preferred embodiment of the present invention, Resulting products made by the methods in Claims 1-21.

### DETAILED DESCRIPTION OF THE INVENTION

The present invention provides unique and novel beta vulgaris-based products as well as methods for producing the products that preserve the therapeutic influence of the components contained in the Beta vulgaris and especially in red beet species. Red beet contains substances that had been shown to have therapeutic effect on the body. It is important to preserve the activity of the components when producing the products. In addition, red beet has side tastes that are not favorable when eating the product itself and the methods of producing the products of the present invention eliminate the unfavorable taste of the red beet so as to allow producing products such as beverages that are tasty and can be consumes by many populations.

The present invention provides a main aspect by which a juice made of red beet is produces wherein the juice can be produces from the root, pilled or un-pilled, from the leaves or from both. It is then being sweetened so as to eliminate any bad taste and can be fermented so as to establish an alcoholic beverage. Moreover, fermentation is known to increase the biological availability of active components in the body therefore, fermenting the red beet should increase the biological availability of the specific active components in the red beet to the body. The juice can be filtered and used for other products. Following are examples of producing red beet based products.

### EXAMPLE 1

Stages in the Preparation of Alcoholic Beverage:

Preparing juice from a red beet root (with or without the peel): Extracting the juice from the beet using a juice extractor, for example. The red beet can be slightly heated so as to facilitate the peeling of the peel in case peeling is desired.

Adding sugar based component to the juice to facilitate fermentation. The sugar based component can be chosen of a group comprising fruit extract (preferably apple extract), beet molasses, sugar cane molasses, honey, maltose, brown or white sugar, sweet pomegranate extract. The percentage of sugar component depends on the final alcohol level that is required for the beverage. It is preferable that the final amount of sugar content in the mixture will be substantially 25% so that the final percentage of alcohol will stand substantially on 12%.

Adding yeasts in case non-natural fermentation is desired.

Fermenting the resulting mixture.

Filtering the mixture so as to obtain the fluid.

It is optional, however preferable to maintain the mixture in an oak barrel so as to improve the flavor and aroma of the beverage.

Optionally, the fermentation process can be extended to a malolactic fermentation as well.

Other materials can be added into the beverage such as spices and medical herbs so as to improve the taste and the quality of the beverage. Optionally, chips of oak tree can be also added after the fermentation process is finished. Whisky or other alcoholic drinks can be also added.

In order to produce a sweet beverage, oligofructose or oligosaccharides can be added. Any other additives that are added into the red beet root based alcoholic beverage are covered by the scope of the present invention.

Based on this preparation, vinegar or seasoning product can be produced.

It should be mentioned that the alcoholic beverage is a preferable beverage due to the importance of alcohol consumption in relative low quantities on well being. It had been shown that moderate consumption of alcohol has a protective effect over the heart. The increase in homocysteine in the blood that might be also a consequence of alcohol consumption is eliminated due to the fact that the red beet contains also folic acid, vitamin B6, and betain that cause an opposing effect on homocysteine as mentioned herein before.

An additional reason for the preference of alcoholic beverages is that the fermentation process increases the biological availability of certain components. Alcohol also decreases stress and gives a synergistic effect with the active components in the red beet, which are triterpen saponins that provides the red beet with adaptogenic activity. Stress by itself imparts risk of atherosclerosis so this risk is also reduced due to drinking the red beet-based alcoholic drink of the present invention.

Since alcohol can have a negative effect on the liver, incorporating the alcohol with the red beet components that are known to have a protective effect on the liver, as mentioned herein before, is preferable.

### EXAMPLE 2

The stages for the production of vinegar or seasoning are as follows:

Preparing juice from a red beet root (with or without the peel).

Adding sugar based component to the juice for fermentation. Optionally, the fermentation process can be based on the natural content of sugar that is present in the beet itself. If sugar from another source is added, the percentage of sugar should not exceed 18%.

Adding yeasts in case non-natural fermentation is desired.

Fermenting the resulting mixture.

allowing natural acidation.

Filtering the mixture so as to obtain the fluids.

Adding bacteria (such as acetic acid bacteria) so as to sour the liquid.

### EXAMPLE 3

Another product based on the root of a red beet is a jelly or delicacy.

The stages for preparing a jelly comprises:

Preparing a juice from a red beet root (with or without the peel).

Adding sugar based component or sugar substitute. The sugar based component can be chosen of a group comprising fruit extract (preferably apple extract), beet molasses, sugar cane molasses, honey, maltose, brown or white sugar, oligofructose, oligosaccharide, or stevioside. Oligofructose and oligosaccharide are preferred in gelly based foods. Adding the sugar can be performed after other stages as well, so as to improve the taste.

Heating the mixture.

Adding lemon juice.

Adding gelling additive. The gelling substance can be chosen from a group consisting of agar, tragacanth gum, gelatine, or pectin. Any other additive that forms gelation is covered by the scope of the present invention.

Steering the mixture until the mixture is fully homogenous.

Cooling the mixture.

This product can be improved by sweetening components, spices or herbs.

This product is adapted for reducing the cholesterol and the homocysteine levels in the blood as well as improving the functionality of the liver. The gelling product can also improve and enhance the intestinal activity, to reduce the appetite, and to reduce markedly the risks for large intestine cancer, especially when sweetened with oligofructose or oligosaccharides.

### EXAMPLE 4

An additional beverage that can be produces from red beet is a juice. The preparation comprises the following stages:

Preparing juice from a red beet root (with or without the peel).

Adding sweetening agent.

Optionally, adding carbon dioxide for producing a gaseous beverage.

Optionally, adding spices or herbs or food supplements.

### EXAMPLE 5

A dry extract from red beet can be also produced in accordance to the present invention. The preparation comprises the following stages:

Preparing a juice from a red beet root (with or without the peel).

Drying the juice into a powder. Drying the juice can be performed in a freeze drying procedure, or spray drying.

Encapsulating the powder into pills, for example.

The powder is adapted for reducing the cholesterol and the homocysteine levels in the blood.

It should be noted that incorporating medical herbs into the powder in any form can have an effect of increased blood count. This specific formulation of red beet dry extract and medical herbs, which may be for example dry Astragalus membranaceus extract and dry withania somniferum extract, can have a tremendous influence as a supportive treatment on cancer patients that are being treated in a chemotherapeutic procedure.

### EXAMPLE 6

An additional product based on red beet juice or squash is a paste. The paste preparation comprises the following stages:

Preparing juice from a red beet root (with or without the peel).

Adding olive oil.

Adding lecithin.

The paste can be used for spreading on bread, for example.

### EXAMPLE 7

A composition especially for woman with gynecologic problems such as menopause, PMS, and fertility problems, is produced through from the following components:

Dry red beet extract.

Dry pomegranate extract.

Additional additives such as Cimicifuga racemosa, fatty acid—gla, ferulic acid, Chamaelirium luteum, and Vitex agnus castus.

As mentioned herein before, other additives can be added into the mixtures that produce the different products as disclosed herein. Examples for herbs that can be added separately or more than one herb are as follows: Silybum marianum, Taraxacum spp., Curcuma spp., Cynara scolymus, Commiphora mukul, Ganoderma lucida, Gymnema sylvestre, Allium sativum, Trigonella foenum-graecum, Atriplex spp., Pterocarpus marsupium, Momordica chrantia, Vaccinium myrtillus, Panax spp., Vanilla spp. Coriandrum sativum. Piper nugrum, Gentiana spp.

Examples for food additives are as follows: B-complex vitamins such as folic acid, B12, B6, and niacin, Omega 3 fatty acids, chromium, vitamin C, camitine, inulin.

Eliminating the possible negative effects of the red beet can be established by adding vitamin C so as to prevent formation of nitrosamines. Oxalic acid can be also eliminated. The natural sugar present in the red beet is eliminated in case the final product is for low calories dietary product.

Optionally, the products can go through a sterilization process during any one of the stages.

The beverages (alcoholic, nonalcoholic, gaseous) can optionally be mixed with juices prepared from pomegranate. The pomegranate juice can be mixed in any of the stages.

While the invention has been described with reference to certain exemplary embodiments, various modifications will be readily apparent to and may be readily accomplished by persons skilled in the art without departing from the spirit and scope of the above teachings.

It should be noted that these products can be also applied in shampoo products as well as soaps so as to treat skin diseases such as seborrhea and seborrheic dermatitis. Other products for external use can be provided with extracts of the red beet and food supplements such as B6, zinc, selenium, medical herbs such as Arctum lappa, Calendula officinalis, Aloe spp. and Urtica spp, Tabebuia spp., Echinacea spp, Salix spp.

It should be also noted that the use of red beet is preferable, however, the products mentioned herein and used in the present invention can be produced and prepared from other beta vulgaris varieties.

It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims. The terms “comprise”, “include” and their conjugates as used herein mean “include but are not necessarily limited to”.

### REFERENCES

Bobek P et al. The effect of red beet (Beta vulgaris var. rubra) fiber on alimentary hypercholesterolemia and chemically induced colon carcinogenesis in rats. Nahrung, 2000; 44(3): 18⁴-187 p.
Hagander B et al. Dietary fibre enrichment, blood pressure, lipoprotein profile and gut hormons in NIDDM patients. Eur J Clin Nutr 1989; 43: 35-44 p.
Jones P. J. H et al. Cholesterol-lowering efficacy of a sitostanol-containing phytosterol mixture with a prudent diet un hyperlipidemic men. Am J Clin Nutr 1999; 69: 1144-1150 p.
Kahkonen M. P et al. Antioxidant activity of plant extracts containing phenolic compounds. J Agric Food Chem 1999; 47: 3954-3962 p.
Krispil N. Beta vulgaris (Red beet). Medical plants in Israel and through out the world: The complete guide 2000: 170-171 p. (the book in Hebrew) [a]
Krispil N. Beta vulgaris (beet). Medical plants in Israel and through out the world: The complete guide 2000: 171 p. (the book in Hebrew) [b]
Kujala T. S et al. phenolics and betacyanines in red beetroot (Beta vulgaris) root: distribution and effect of cold storage on the content of total phenolics and three individual compounds. J Agric Food Chem 2000; 48:5338-5342 p.
Lalouschek W et al. Hyperhomocyst(e)inemia—an independent risk factor of stroke, Fortschr Neurol Psychiatr 1996; 64(7): 271-277 p.
PDR for herbal monographs. Beta Vulgaris. 1^{th} Edition 1999: 505-523 p.
Sella B. A & Dolman R. Dialysis attended and organs transplanted that consume Cyclosporin: tow sectors of patients with high levels of Homocystein, Harefua 1999; 136(3): 218-222 p. (the article in Hebrew)
Shils M. E et al. Nutrition and the immune system, 9^{th} Edition. Modern nutrition in health and disease 1999: 741-742 p.
Weiss R. F. Beta vulgaris (Sugar Beet). Herbal Medicine 1988: 89 p.

1. A method of reducing homocysteine levels as well as reducing the cholesterol levels in the blood among other therapeutic effects by orally administering a subject with beta vulgaris-based composition prepared by a process comprising preparing juice from beta vulgaris root.

2. The method as claimed in claim 1, wherein the process further comprising fermenting the juice.

3. The method as claimed in claim 1, further comprising drying said juice into powder.

4. The method as claimed in claim 1, wherein said beta vulgaris is red beet.

5. The method as claimed in claim 1, wherein said sweetening agent is selected from a group comprising fruit extract (preferably apple extract), beet molasses, sugar cane molasses, honey, maltose, brown or white sugar, sweet pomegranate extract, oligofructose, oligosaccharides.

6. The method as claimed in claim 1, wherein the process further comprising:
adding oil;
adding lechitin;
so as to produce paste adapted for spreading.

7. The method as claimed in claim 1, wherein the process further comprising:
adding medical herbs.

8. A method of reducing effects of gynecologic problems such as menopause, PMS, and fertility problems by orally administering a female with red beet-based composition comprising:
dry red beet extract;
dry pomegranate extract;
additives selected of a group comprising Cimicifuga racemosa, fatty acid—gla, ferulic acid, Chamaelirium luteum, and Vitex agnus castus.

9. A method of increasing the blood count of a subject, especially a subject going through a chemotherapy treatment, by orally administering the subject with red beet-based composition comprising:
dry red beet extract;
dry Astragalus membranaceus extract.

10. A method of increasing the blood count of a subject, especially a subject going through a chemotherapy treatment, by orally administering the subject with red beet-based composition comprising:
dry red beet extract;
dry withania somniferum extract.

11. A method of treating skin diseases such as seborrhea and seborrheic dermatitis by externally spreading the subject with products adapted for external use comprising juice extracted from beta vulgaris root.

12. A method as claimed in claim 13, wherein said beta vulgaris is a red beet.
